(19) United States
(12) Patent Application Publication
(10) Pub. No.: US 2024/0262809 A1
(43) Pub. Date: Aug. 8, 2024

(54) METHOD FOR PREPARING INTERMEDIATE FOR SYNTHESIS OF XANTHINE OXIDASE INHIBITOR

(71) Applicant: LG CHEM, LTD., Seoul (KR)

(72) Inventors: In Ae RYU, Seoul (KR); Ju Young LEE, Cheongju-si (KR); Joo Yong YOON, Seoul (KR); Seok Ju LEE, Seoul (KR); Ah Byeol PARK, Seoul (KR); Ki Dae KIM, Seoul (KR); Hui Rak JEONG, Seoul (KR)

(73) Assignee: LG CHEM, LTD., Seoul (KR)

(21) Appl. No.: 18/557,775

(22) PCT Filed: Apr. 26, 2022
(86) PCT No.: PCT/KR2022/005925

§ 371 Date: Oct. 27, 2023

(30) Foreign Application Priority Data
Apr. 27, 2021 (KR) 10-2021-0054150

(51) Int. Cl. C07D 403/10

(52) U.S. Cl. CPC C07D 403/10

(57) ABSTRACT

The present invention relates to a novel method for preparing an intermediate of Chemical Formula 2, wherein the intermediate can be effectively used in the synthesis of a xanthine oxidase inhibitor.

### TECHNICAL FIELD

The present invention relates to a method for preparing a key intermediate for the synthesis of a xanthine oxidase inhibitor, more specifically to a new method for preparing an intermediate of the following Chemical Formula 2 using an inexpensive starting material and a C—N coupling reaction:

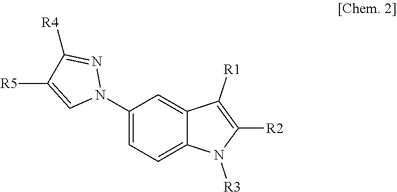

wherein
R1 is hydrogen;
R2 is hydrogen, halogen, C₁-C₇ alkyl, C₁-C₇ alkoxy-C₁-C₇ alkyl or phenyl;
R3 is hydrogen; C₁-C₇ alkyl unsubstituted or substituted with a substituent selected from halogen, C₃-C₇ cycloalkyl or O—R6, wherein R6 represents C₁-C₄ alkyl; C₃-C₇ cycloalkyl; or

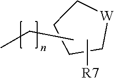

(wherein W represents O or S, R7 represents hydrogen or

C₁-C₄ alkyl, and n is an integer 0 to 3);
R4 is hydrogen, halogen or C₁-C₇ alkyl; and
R5 is —C(O)OR8, where R8 is hydrogen, C₁-C₇ alkyl or C₃-C₇ cycloalkyl.

### BACKGROUND ART

Xanthine oxidase is known as an enzyme that converts hypoxanthine to xanthine and the formed xanthine to uric acid. Since uricase, which exists in most mammals, does not exist in humans and chimpanzees, a substance called uric acid is known to be the final product of purine metabolism (S. P. Bruce, Ann. Pharm., 2006, 40, 2187-2194). Uric acid contained in the blood at high concentrations causes various diseases, a representative example of which is gout.

As mentioned above, gout is a disease caused by high levels of uric acid in the body, and refers to a condition in which uric acid crystals accumulate in joint cartilage, ligaments, and surrounding tissues, causing severe inflammation and pain. Gout is a type of inflammatory joint disease, the incidence of which has been steadily increasing over the past 40 years (N. L. Edwards, Arthritis & Rheumatism, 2008, 58, 2587-2590).

Looking at the number of patients with gout in Western countries from the 1960s to the mid-1990s, there is a surprising increase of about 200% to 300%, and patients with gout are mainly men. Obesity, aging, decreased kidney function, high blood pressure, and the like are considered to be the causes of the rate of increase in patients with gout. The incidence of gout is about 1.4/1000 people, but also vary depending on the level of uric acid. In other words, the incidence of gout in patients having a uric acid level in the blood of 7.0 mg/dl or more is 0.5%, but the incidence of gout in patients having a uric acid level in the blood of 9.0 mg/dl or more is 5.5% (G. Nuki, Medicine, 2006, 34, 417-423). Considering the incidence, it can be seen that uric acid concentration in the blood is an important factor causing gout. Additionally, dietary habits, alcohol, lipids, obesity, and the like may also act as important factors causing gout. These days, studies on the correlation between uric acid and heart failure, high blood pressure, diabetes, kidney disease, and cardiovascular disease are being actively carried out by a number of researchers, and the importance of uric acid management is increasing (D. I. Feig et al., N. Eng. J. Med, 2008, 23, 1811-1821). In addition, allopurinol, a xanthine oxidase inhibitor, is known to be effective in ulcerative colitis (Aliment. Pharmacol. Ther. 2000, 14, 1159-1162; WO 2007/043457).

Until febuxostat was approved as an arthrifuge in the United States in 2009 (Brain Tomlinson, Current opin. invest. drugs, 2005, 6, 1168-1178), allopurinol was the only drug used to treat gout for the past 40 years. Allopurinol is known as a non-specific inhibitor of various enzymes involved in purine and pyrimidine metabolism, and has a Ki of 700 nM against xanthine oxidase (Y. Takano et al., Life Sciences, 2005, 76, 1835-1847). Allopurinol is directly oxidized by xanthine oxidase and converted to oxypurinol, and this metabolite is known to act as a greatly strong inhibitor of xanthine oxidase.

However, allopurinol is known to cause gastrointestinal side effects and skin rashes, and to have poor compliance when taken for a long period of time. In particular, among patients taking allopurinol, it has been reported that an unpredictable fatal side effect of Stevens-Johnson syndrome occurs at a low ratio (Felix Arellano et al, Ann. Pharm., 1993, 27, 337-43). This side effect is known to be a serious side effect that causes cell necrosis in the skin and mucous membranes of the mouth, leading to death in about 25% of cases if not treated appropriately.

Accordingly, various studies have been conducted to develop new xanthine oxidase inhibitors, and Korean Patent Publication No. 10-2011-0037883 discloses a novel compound of the following Chemical Formula 1, which is effective as a xanthine oxidase inhibitor:

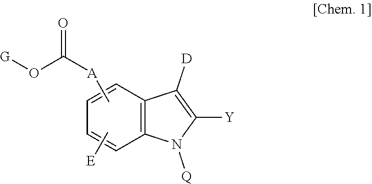

in Chemical Formula 1,
A is selected from the following substituents A-i, A-ii, A-iii, A-iv, A-v, A-vi, A-vii and A-viii,

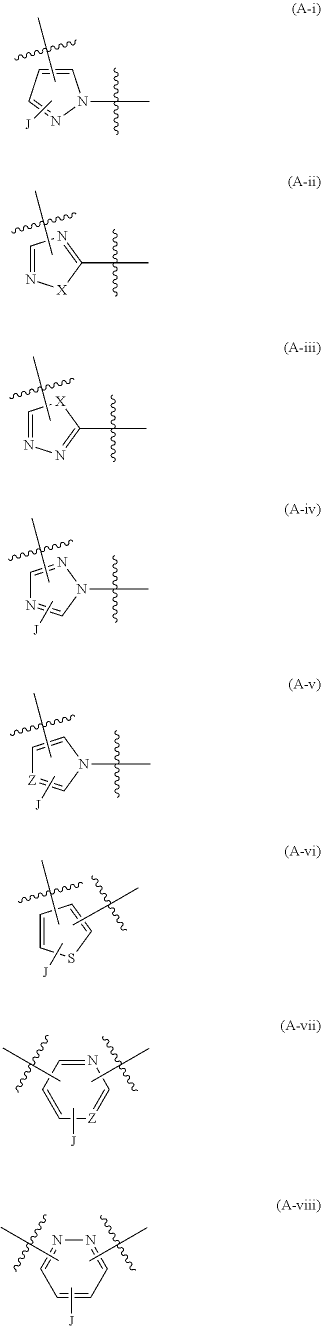

wherein
J represents hydrogen, halogen, or C₁-C₆-alkyl unsubstituted or substituted with halogen,
X is O or S, and
Z is C or N,
E represents hydrogen, halogen, cyano, nitro, substituted or unsubstituted C₁-C₆-alkyl, or substituted or unsubstituted C₁-C₆-alkoxy,
D represents hydrogen, halogen, cyano, nitro, C₁-C₆-alkyl unsubstituted or substituted with halogen, —CHO, or —CH═N—OH,
Q is selected from the following substituents Q-i, Q-ii, and Q-iii-1 to Q-iii-9
(Q-i) hydrogen;
(Q-ii) substituted or unsubstituted linear, branched or cyclic, saturated or unsaturated alkyl;

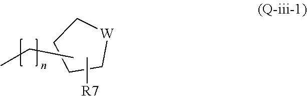

(wherein W represents O or S, R7 represents hydrogen or substituted or unsubstituted lower alkyl, and n is an integer 0 to 3);

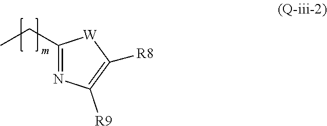

(wherein W represents O or S, R8 and R9 each independently represent hydrogen or lower alkyl, and m is an integer 1 to 3);

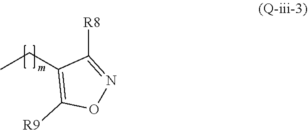

(wherein R8 and R9 each independently represent hydrogen or lower alkyl, and m is an integer 1 to 3);

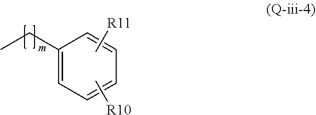

(wherein, R10 and R11 each independently represent hydrogen, halogen, lower alkoxy, or lower alkyl, and m is an integer 1 to 3);

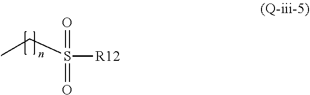

(wherein R12 represents substituted or unsubstituted lower alkyl or aromatic, and n is an integer 0 to 3);

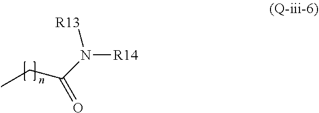

(wherein, R13 and R14 each independently represent substituted or unsubstituted lower alkyl, or may form a 3- to 7-membered heterocycle containing N, and n is an integer 0 to 3);

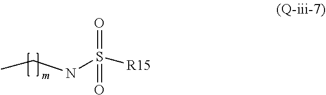

(wherein R15 represents substituted or unsubstituted lower alkyl, and m is an integer 1 to 3),

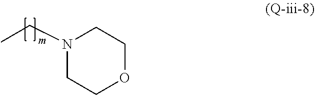

(wherein m is an integer 1 to 3); and

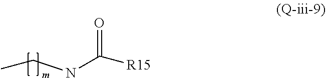

(wherein R15 represents substituted or unsubstituted lower alkyl, and m is an integer 1 to 3),
Y represents hydrogen, halogen, substituted or unsubstituted linear, branched or cyclic saturated or unsaturated alkyl, substituted or unsubstituted C₁-C₆-alkoxy, substituted or unsubstituted aromatic, or heteroaromatic, and
G represents hydrogen or substituted or unsubstituted linear, branched or cyclic, saturated or unsaturated alkyl.

In a specific example of the document, the preparation of 1-(3-cyano-1-isopropyl-indol-5-yl)pyrazole-4-carboxylic acid according to the following Scheme 1 is disclosed.

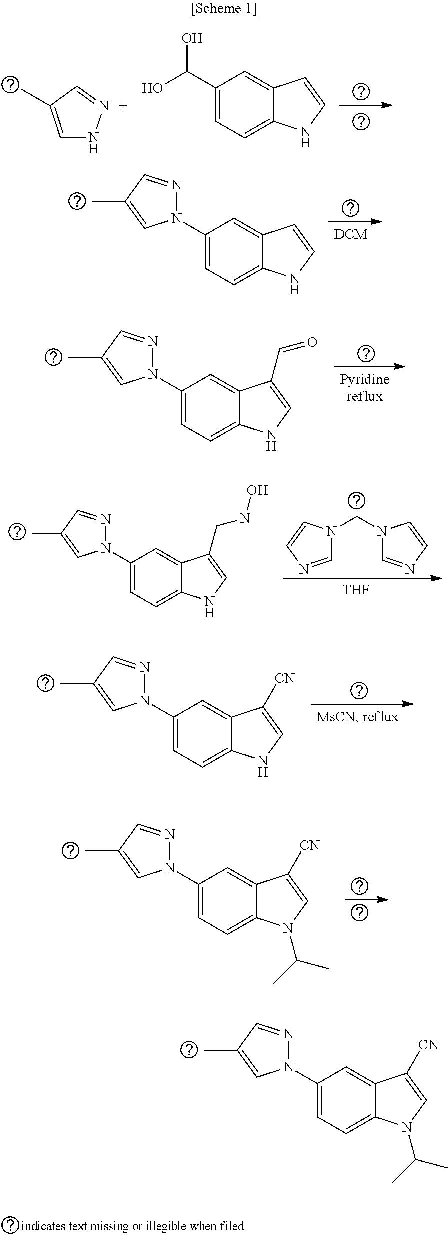

In the first step of Scheme 1, 1H-pyrazole-4-carboxylic acid ethyl ester and 1H-indol-5-ylboronic acid are dissolved in N,N-dimethylformamide (DMF), copper(II) acetate and pyridine are added, and the mixture is stirred at room temperature for 3 days to prepare 1-(1H-indol-5-yl)pyrazole-4-carboxylic acid ethyl ester.

However, in the method, indolylboronic acid is used as a starting material, but this material is an expensive material, and there is a report that 1-(3-cyano-1-isopropyl-indol-5-yl)pyrazole-4-carboxylic acid is obtained at a yield of 77% when this material is reacted with 1 g of 1H-pyrazole-4-carboxylic acid ethyl ester as well as that the yield decreases to 50% when the compound is scaled up to 18.4 g, so there is a problem that this method is not preferable to be applied to a scale up process.

### SUMMARY OF INVENTION

### Technical Problem

Accordingly, the technical object of the present invention is to provide a method suitable for mass production of a compound of Chemical Formula 2, which is a key intermediate in the synthesis of an excellent xanthine oxidase inhibitor, at lower cost.

### Solution to Problem

In order to achieve the object, the present invention provides a method for preparing a compound of Chemical Formula 2 by conducting a C—N coupling reaction of a compound of Chemical Formula 3 with a compound of Chemical Formula 4 in the presence of a copper catalyst, a base, and a ligand in an organic solvent.

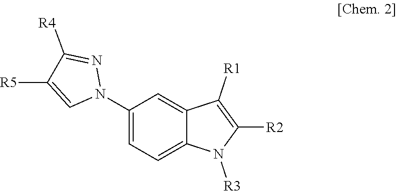

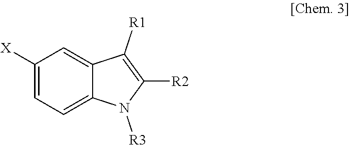

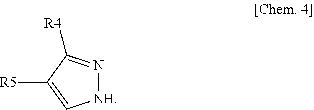

in the chemical formulas,
X is F, Cl, Br or I,
R1 is hydrogen;
R2 is hydrogen, halogen, C₁-C₇ alkyl, C₁-C₇ alkoxy-C₁-C₇ alkyl or phenyl;
R3 is hydrogen; C₁-C₇ alkyl unsubstituted or substituted with a substituent selected from halogen, C₃-C₇ cycloalkyl or O—R6, wherein R6 represents C₁-C₄ alkyl; C₃-C₇ cycloalkyl; or

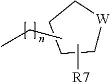

(wherein W represents O or S, R7 represents hydrogen or C₁-C₄ alkyl, and n is an integer 0 to 3);
R4 is hydrogen, halogen or C₁-C₇ alkyl; and
R5 is —C(O)OR8, where R8 is hydrogen, C₁-C₇ alkyl or C₃-C₇ cycloalkyl.

Hereinafter, the present invention will be described in detail.

In the present invention, an intermediate compound of Chemical Formula 2 is synthesized through a C—N coupling reaction of a compound of Chemical Formula 3 with a compound of Chemical Formula 4.

In an embodiment according to the present invention, as the organic solvent in the method, for example, one or more selected from xylene, toluene, dimethylformamide (DMF), or dimethyl sulfoxide (DMSO) may be used.

In another embodiment according to the present invention, as the copper catalyst in the method, for example, one or more selected from CuI, Cu(OAc)₂, Cu, Cu₂O or CuO may be used.

In an embodiment according to the present invention, as the base in the method, for example, one or more selected from potassium carbonate (K₂CO₃), cesium carbonate (Cs₂CO₃), potassium phosphate tribasic (K₃PO₄), triethylamine (Et₃N) or sodium tert-butoxide (NaOtBu) may be used.

In an embodiment according to the present invention, as the ligand in the method, for example, one or more selected from 1,2-cyclohexanediamine, N,N′-dimethyl-1,2-cyclohexanediamine, N,N′-dimethylethylenediamine, 1,10-phenanthroline, proline, an oxime ligand or a tetradentate ligand may be used.

### Advantageous Effects of Invention

In the preparation method of the present invention, since a compound of Chemical Formula 3, which is commercially easily purchased in large quantities, is introduced, and the process is simplified so that scale up is possible, an intermediate of Chemical Formula 2 can be mass-produced at a high yield.

### DESCRIPTION OF EMBODIMENTS

Hereinafter, the present invention will be described in more detail with reference to Example. However, the following Example is merely illustrative to aid understanding of the present invention, and the scope of the present invention is not limited thereto.

### Example: Synthesis of 1-(1H-indol-5-yl)pyrazole-4-carboxylic acid ethyl ester

1H-pyrazole-4-carboxylic acid ethyl ester (14.3 g, 102 mmol) and 5-bromo-1H-indole (20 g, 102 mmol) were added to 120 ml of xylene. CuI, 1,2-cyclohexanediamine, and K₂CO₃ were added thereto, and the mixture was stirred under reflux for 20 hours. The solvent was distilled off under reduced pressure, ethyl acetate (EtOAc) was added, washing with NH₄OH aqueous solution was performed, and the organic layer was filtered through Na₂SO₄/silica gel. The solvent was distilled off under reduced pressure, and crystallization with toluene/n-hexane was performed to obtain 22.3 g (85% yield) of the title compound.

¹H-NMR (CDCl₃) δ 8.39 (1H, s), 8.33 (1H, Br), 8.11 (1H, s), 7.91 (1H, d), 7.53 (1H, dd), 7.47 (1H, d), 7.31 (1H, t), 6.63-6.62 (1H, m), 4.35 (2H, q), 1.39 (3H, t)

1. A method for preparing a compound of Chemical Formula 2, the method comprising conducting a C—N coupling reaction of a compound of Chemical Formula 3 with a compound of Chemical Formula 4 in presence of a copper catalyst, a base, and a ligand in an organic solvent: 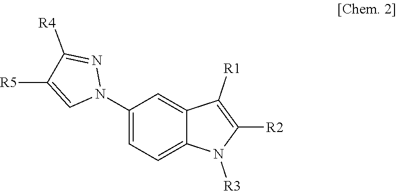 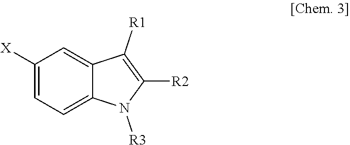 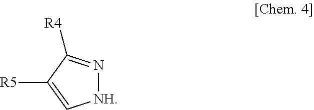
in the chemical formulas,
X is F, Cl, Br or I,
R1 is hydrogen;
R2 is hydrogen, halogen, C₁-C₇ alkyl, C₁-C₇ alkoxy-C₁-C₇ alkyl or phenyl;
R3 is hydrogen; C₁-C₇ alkyl unsubstituted or substituted with a substituent selected from halogen, C₃-C₇ cycloalkyl or O—R6, wherein R6 represents C₁-C₄ alkyl; C₃-C₇ cycloalkyl; or 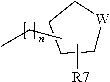
(wherein W represents O or S, R7 represents hydrogen or C₁-C₄ alkyl, and n is an integer 0 to 3);
R4 is hydrogen, halogen or C₁-C₇ alkyl; and
R5 is —C(O)OR8, wherein R8 is hydrogen, C₁-C₇ alkyl or C₃-C₇ cycloalkyl.

2. The preparation method according to claim 1, wherein the organic solvent is one or more selected from xylene, toluene, dimethylformamide (DMF), or dimethyl sulfoxide (DMSO).

3. The preparation method according to claim 1, wherein the copper catalyst is one or more selected from CuI, Cu(OAc)₂, Cu, Cu₂O or CuO.

4. The preparation method according to claim 1, wherein the base is one or more selected from potassium carbonate, cesium carbonate, potassium phosphate tribasic, triethylamine or sodium tert-butoxide.

5. The preparation method according to claim 1, wherein the ligand is one or more selected from 1,2-cyclohexanediamine, N,N′-dimethyl-1,2-cyclohexanediamine, N,N′-dimethylethylenediamine, 1,10-phenanthroline, proline, an oxime ligand or a tetradentate ligand.
